(19) United States
(12) Patent Application Publication
(10) Pub. No.: US 2019/0249362 A1
(43) Pub. Date: Aug. 15, 2019

(54) COMPOSITION FOR THE AEROBIC TREATMENT OF LIGNOCELLULOSIC RESIDUES, ASSOCIATED METHOD AND USE OF SAME FOR BIOLOGICAL PRETREATMENT OF SAID RESIDUES

(71) Applicant: Institut national de Recherche en Sciences et Technologies pour I'Environnement et I'Agriculture (IR, ANTONY (FR)

(72) Inventors: Pascal PEU, RENNES (FR); Jianghao TIAN, MASSY (FR); Anne-Marie POURCHER, SAINT GREGOIRE (FR)

(21) Appl. No.: 16/342,000

(22) PCT Filed: Oct. 16, 2017
(86) PCT No.: PCT/FR2017/052841

§ 371 Date: Apr. 15, 2019

(30) Foreign Application Priority Data
Oct. 20, 2016 (FR) 1660188

(51) Int. Cl. D21B 1/02; C12P 19/14; C12P 7/10

(52) U.S. Cl. CPC D21B 1/021; C12P 2201/00; C12P 7/10; C12P 19/14

(57) ABSTRACT

A composition for treating lignocellulosic agricultural residues (11), such as straw, characterised in that it comprises a liquid mixture of an aqueous solution containing the mineral elements NPK, capable of forming a nutrient medium for endogenous microorganisms of said agricultural residues to be treated, and a source of lignin obtained from a process of alkaline delignification of lignocellulosic biomasses. Treating said residues in an aerobic medium with the composition increases the accessibility of the cellulose to cellulases. Use for the biological pretreatment of said lignocellulosic residues.

### FIELD OF THE INVENTION

The present invention relates to the field of the degradation of agricultural residues, and more particularly of the degradation of lignocellulosic agricultural residues such as straw.

The invention also relates to a composition making it possible to degrade these lignocellulosic residues, a treatment process employing such a composition, and also the use thereof as biological treatment of said residues.

### PRIOR ART

At the present time, agricultural plant residues are being increasingly upgraded in processes of anaerobic digestion, leading to the production of a biogas comprising methane.

Among these crop residues, lignocellulosic residues are among the slowest to be degraded during anaerobic digestion. This is because this lignocellulosic biomass comprises on the one hand cellulose fibers and hemicellulose fibers, forming holocellulose, and on the other hand a matrix based on lignin, a heteropolymer which constitutes a protective barrier for the cellulose and hemicellulose fibers. Thus, the anaerobic biodegradation of straw (which is lignocellulosic residue) in digesters (to produce methane) may be slow and exceed 40 days. For some cereal straws, such as rapeseed straw, anaerobic biodegradation is even slower.

Moreover, this rapeseed straw is very rarely used as bedding or feed for animals and is commonly left in the fields after the cereals have been harvested. This straw therefore constitutes an abundantly available biomass which would be useful to upgrade.

There is therefore a need to pretreat this lignocellulosic biomass, in particular before introducing it into anaerobic digesters.

### Aims

A first aim of the invention is therefore to propose a process for treating these lignocellulosic residues in order to facilitate or accelerate the digestion thereof in anaerobic digesters such as methanizers.

Another aim of the invention is to propose an agent and a process making it possible to destructure the lignin-based matrix of these lignocellulosic residues, with a view to increasing the accessibility of the cellulosic fibers, consequently enabling degradation of the cellulosic fibers released in this way.

Different types of processes for destructuring lignocellulosic biomass are known to date:
physical processes such as milling, heating, pressure, ultrasonication, microwave irradiation, or else a combination of these processes leading to destructuring of the lignocellulosic matrix, enabling easier release of fermentible monosaccharides;
chemical processes: acid or alkaline, or by means of organic solvents, or else by means of powerful oxidizing agent such as ozone or a combination of these chemical processes;
biological processes by specific microorganisms or enzymes.

The inventors focused on the latter category of processes, due to the following advantages: (i) they require minimal energy investment, (ii) are inexpensive and (iii) have specificity of action.

With a view to developing such a biological treatment, the inventors, during their research of isolating microorganism strains that may have lignolytic capacity, surprisingly discovered a composition that makes it possible to significantly increase the accessibility of the cellulose of the lignocellulosic residues to cellulases, and therefore to effectively degrade such residues. It has also proved that such a composition was in particular effective with regard to rapeseed straw residues.

### DETAILED DESCRIPTION

The present invention therefore relates to a composition for the treatment of agricultural plant residues, especially lignocellulosic residues, of straw type, characterized in that it comprises a liquid mixture of an aqueous solution containing NPK mineral salts, able to form a nutritive medium for endogenous microorganisms of said agricultural residues to be treated, and of a source of lignin originating from a process of alkaline delignification of lignocellulosic biomasses.

Advantageously, the source of lignin is lignin originating from the Kraft process of paper pulp industries, referred to as Kraft lignin.

The examples presented below show synergy between the NPK nutritive salts and the Kraft lignin to increase the accessibility of the cellulose of lignocellulosic residues to cellulases.

The concentration of lignin in said liquid mixture is preferably between 0.1 g/l and 3 g/l, preferably between 1 g/l and 2.5 g/l, even more preferably between 2 g/l and 2.5 g/l. The lignin, preferably the Kraft lignin, is advantageously added into the solution of NPK mineral salts.

Regarding the aqueous solution containing the NPK mineral salts, it preferably comprises from 10 to 300 mmol/l of potassium K, from 20 to 200 mmol/l of phosphorus P and from 5 to 100 mmol/l of nitrogen N.

The aqueous solution containing the NPK mineral salts comprises the molar ratios N:P of advantageously between 1:10 and 1:1 and P:K of between 1:5and 10:1.

Preferably, in the aqueous solution containing the NPK mineral salts, nitrogen N is present in the form of ammonium ions and phosphorus P in the form of phosphate ions.

The present invention also relates to the process for treating agricultural plant residues, especially lignocellulosic residues, of straw type, characterized in that it comprises a step of treatment by bringing said lignocellulosic residue into contact, in aerobic medium, with a composition as described above, said treatment leading to an increase in the accessibility of the cellulose of said lignocellulosic residue to cellulases.

The tests carried out show that this accessibility of the cellulose may be multiplied by between 5 and 8 times, in just three days of aerobic treatment.

The lignocellulosic agricultural plant residues may be chosen from cereal straw such as wheat, rapeseed, corn, barley, oat or rye straw, preferably rapeseed straw.

This straw may be used in crude form, uncut, or cut just to reduce the bulk thereof or to adapt the length thereof to the dimensions of the reactor. It does not therefore require any particular preparation.

The process according to the invention is advantageously carried out in a reactor, in which said lignocellulosic residues are placed, continuously impregnated by said aqueous mixture, having a redox potential of greater than 100 mV, preferably of between 150 mV and 500 mV.

Surprisingly, this aerobic treatment may be carried out solely in the presence of endogenous microorganisms of said agricultural residues to be treated, that is to say without the introduction of microorganisms into the reactor other than those initially present within said residues.

According to an advantageous feature of the process, the ratio of the chemical oxygen demand (COD) of the lignocellulosic residues to the chemical oxygen demand (COD) of the starting aqueous mixture is between 4 and 50, preferably between 5 and 20, more preferably still between 6 and 10.

The composition according to the invention, and also the treatment process described above, may especially be used:
for the biological pretreatment of agricultural plant residues, especially lignocellulosic residues of straw type, with a view to improving the degradation thereof in digesters, such as anaerobic methanization digesters;
for the biological pretreatment of agricultural plant residues, especially lignocellulosic residues of straw type, with a view to improving the degradation thereof in processes for producing bioethanol;
or else for the biological pretreatment of agricultural plant residues, especially lignocellulosic residues of straw type, with a view to improving the degradation thereof in processes for producing simple sugars from lignocellulose.

The process according to the invention thus makes it possible to contribute to upgrading large amounts of lignocellulosic biomass, such as cereal straw, which were hitherto not utilized.

### BRIEF DESCRIPTION OF THE DRAWINGS

The invention will be understood on reading the following description of exemplary embodiments, with reference to the appended drawings, in which:

FIG. 1 is a diagram of a reactor for carrying out the process according to the invention;

FIG. 2 encompasses charts presenting the cellulose accessibility of the different agents tested on rapeseed straw;

and FIG. 3 shows the relative proportions of the different fractions of the lignocellulosic residues after the various treatments tested (11 days): soluble fractions, cellulose, hemicellulose and lignin (Van Soest method).

### EXAMPLES

### Example 1: Composition

An example of composition according to the present invention is given by the mixture comprising the following constituents:
Kraft lignin (supplier Sigma-Aldrich, reference 370959): 2.5 g/l
mixture of nutritive salts, referred to as M9 (supplier Sigma-Aldrich, reference M6030), comprising the following NKP mineral salts: Na₂HPO₄:6.8 g/l; KH₂PO₄:3.0 g/l; NaCl: 0.5 g/l; NH4Cl:1.0 g/l
water

The different constituents are introduced as follows: the Kraft lignin is suspended in the aqueous solution prepared after dissolution of all the nutritive salts mentioned above.

### Example 2: Treatment Process

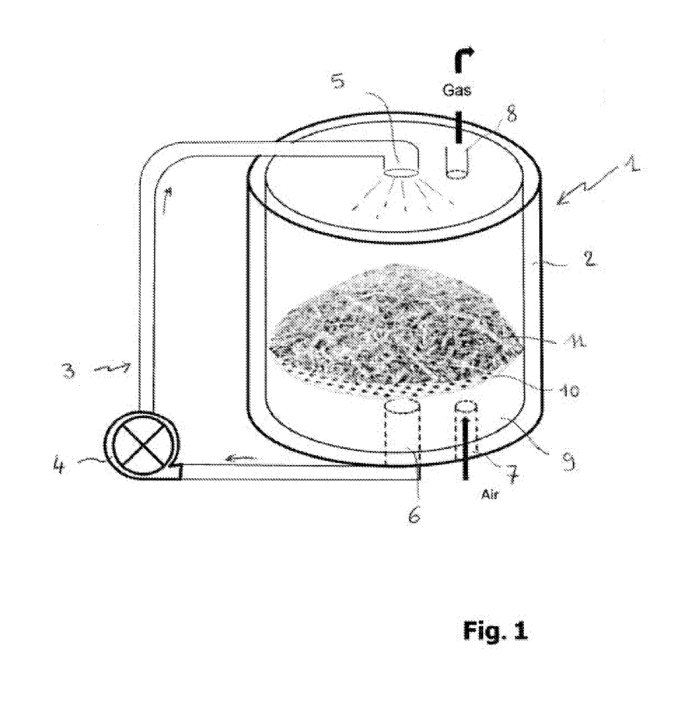

An example reactor used to carry out the process according to the present invention is presented in FIG. 1. The cylindrical reactor 1 has two temperature-controlled walls 2. It is fitted with a circuit 3 for supplying aqueous solution (composition according to the invention of example 1 or other solution to be tested), which supplies said reactor by means of a pump 4. This circuit 3 for supplying aqueous solution comprises, in its upper portion, a spraying nozzle 5 and, in its lower portion, an outlet 6 that takes up the liquid at the bottom 9 of the reactor 1.

The lignocellulosic residues 11 to be treated are placed on a screen 10 arranged a certain distance from the bottom 9 of the reactor. Since the treatment in said reactor 1 takes place in aerobic medium, an injection of air is provided in the bottom 9 of the reactor by means of the air inlet 7. The discharged air and the emitted gases leave the reactor via the outlet 8.

### Operation of the Reactor

The reactor is temperature-controlled to a temperature of between 22 and 38° C., more preferentially of around 30° C. The pH of the solution is neutral (between 6.5 and 7.5). The redox potential of the solution is likewise monitored, and must be at least 100 mV. The solid lignocellulosic residue 11 placed on the screen 10 is permanently impregnated with the aqueous solution, which is continuously sprayed over said residue, but is separated from the stagnant solution at the bottom of the reactor before recycling thereof.

The amount of solution is of course adapted to the amount of lignocellulosic residues: in order to be able to permanently moisten, the ratio of solids of the mass of residue relative to the amount of liquid must not be greater than 2.

The flow rate of the aqueous solution is adjusted to approximately 100 ml per minute. The air is also permanently injected at a flow rate of 25 ml per minute.

The above conditions of flow rate of liquid and of air injected were applied to a reactor with a volume of 3.3 l into which 100 g of rapeseed straw residues were introduced with 400 ml of solution recirculated at a rate of 100 ml per minute.

### Example 3: Comparative

Different tests were carried out with various aqueous nutritive solutions of M9 or NaCl in the presence or absence of Kraft lignin and/or in the presence or absence of lignolytic bacteria:
M9+lignin+Serratia sp.
M9+lignin+Pseudomonas chlororaphis
M9+lignin+Stenotrophomonas sp.
NaCl+lignin+Serratia sp.
NaCl+lignin+Pseudomonas chlororaphis
NaCl+lignin+Stenotrophomonas sp.
M9+lignin
NaCl+lignin
M9
NaCl
   where:
   M9=nutritive solution (see formulation in example 1)

### NaCl=9 g/l

Lignin=Kraft lignin at 2.5 g/l in the nutritive solution or the solution of NaCl
Bacteria=10⁸/ml of solution

Aerobic treatments were carried out for 11 days with measurements respectively on days 3, 5, 7 and 11 of the degree of cellulose accessibility (CAC). This measurement of the accessibility of the residues to cellulase was carried out according to the method of Hong and Zhang (Langmuir, 23, 12535-12540, 2007), on samples immediately after they were taken (not frozen).

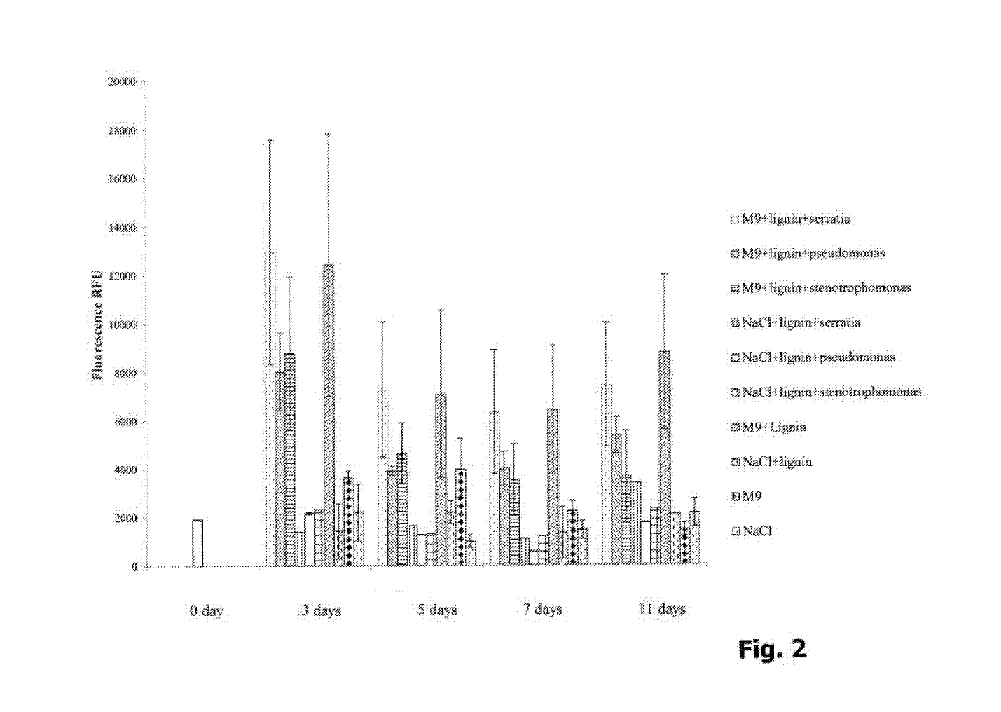

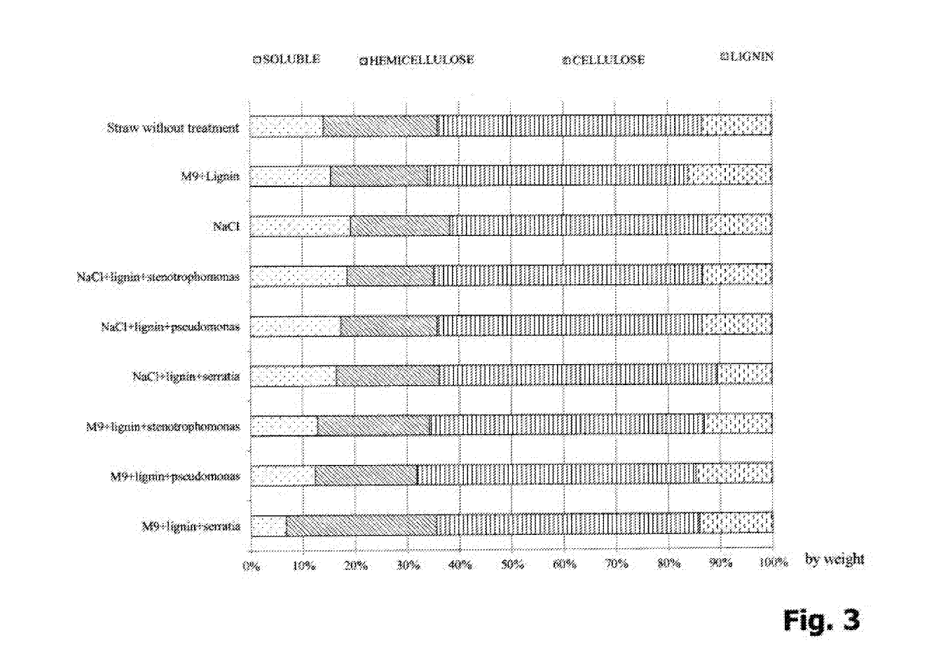

The results obtained are presented in FIG. 2. It is clearly seen that the medium (composition according to the present invention) containing just the nutritive solution M9 and the Kraft lignin enables significant cellulose accessibility of the rapeseed straw.

On the contrary, the bacteria additions made only provide a slight improvement in this parameter of accessibility of the cellulose over the same periods of aerobic treatment. It should also be noted that the composition according to the invention enables a most significant improvement in the cellulose accessibility after just three days of aerobic treatment.

In order to supplement these results, analyses of the different fractions of the lignocellulosic biomass obtained after 11 days of treatment with these different solutions are presented in FIG. 3.

The partitioning was performed using the Van Soest process (Van Soest et al. J Dairy Sci, 74, 3583-3597, 1991). This process, by partitioning the residues chemically using neutral or acidic detergents, makes it possible to determine the respective cellulosic, hemicellulosic or lignin fractions and also the water-soluble fraction.

In light of these results, it is noted that the lignin fraction remains constant whereas the cellulosic and hemicellulosic fractions decrease. The increase in the cellulose accessibility by means of the composition according to the invention is therefore associated with a destructuring of this lignocellulosic biomass, which leads to a release of the cellulose fibers.

This release of the cellulose fibers thus makes it possible to envisage subsequent treatments such as anaerobic treatments, for example digestions with a view to producing methane, simple sugars or else bioethanol.

1. A composition for the treatment of lignocellulosic residues, of straw type, comprising:
a liquid mixture of an aqueous solution containing NPK mineral salts, able to form a nutritive medium for endogenous microorganisms of said agricultural residues to be treated, and of a source of lignin originating from a process of alkaline delignification of lignocellulosic biomasses.

2. The composition as claimed in claim 1, wherein the source of lignin is lignin originating from the Kraft process of paper pulp industries, referred to as kraft lignin.

3. The composition as claimed in claim 1, wherein the concentration of lignin in said liquid mixture is between 0.1 g/1 and 3 g/l.

4. The composition as claimed in claim 1, wherein the aqueous solution containing the NPK mineral salts comprises from 10 to 300 mmol/l of potassium K, from 20 to 200 mmol/l of phosphorus P and from 5 to 100 mmol/l of nitrogen N.

5. The composition as claimed in claim 3, wherein the aqueous solution containing the NPK mineral salts comprises molar ratios of N:P of between 1:10 and 1:1 and P:K of between 1:5 and 10:1.

6. The composition as claimed in claim 1, wherein, in the aqueous solution containing the NPK mineral salts, nitrogen N is present in the form of ammonium ions and phosphorus P in the form of phosphate ions.

7. A process for treating lignocellulosic residues, of straw type, said method comprising the steps of treatment by bringing said lignocellulosic residue into contact, in aerobic medium, with a composition as claimed in claim 1, said treatment leading to an increase in the accessibility of the cellulose of said lignocellulosic residue to cellulases.

8. The process as claimed in claim 7, wherein the lignocellulosic agricultural plant residues are chosen from cereal straw such as wheat, rapeseed, corn, barley, oat or rye straw, preferably rapeseed straw.

9. The process as claimed in claim 7, wherein said process is carried out in a reactor, in which said lignocellulosic residues are placed, continuously impregnated by said aqueous mixture, having a redox potential of greater than 100 mV, preferably of between 150 mV and 500 mV.

10. The process as claimed in claim 7, wherein the treatment is carried out solely in the presence of endogenous microorganisms of said agricultural residues to be treated.

11. The process as claimed in claim 7, wherein the ratio of the chemical oxygen demand (COD) of the lignocellulosic residues to the chemical oxygen demand (COD) of the starting aqueous mixture is between 4 and 50.

12. A process for the biological pretreatment of lignocellulosic residues of straw type, for improving the degradation thereof in anaerobic methanization digesters, said process comprising the step of administering a composition as claimed in claim 1.

13. A process for the biological pretreatment of lignocellulosic residues of straw type, for improving the degradation thereof in processes for producing bioethanol, said process comprising the step of administering a composition as claimed in claim 1.

14. A process for the biological pretreatment of lignocellulosic residues of straw type, for improving the degradation thereof in processes for producing simple sugars from lignocellulose, said process comprising the step of administering a composition as claimed in claim 1.

15. The composition as claimed in claim 3, wherein the concentration of lignin in said liquid mixture is between 1 g/l and 2.5 g/l.

17. The composition as claimed in claim 3, wherein the concentration of lignin in said liquid mixture is between 2 g/l and 2.5 g/l.

18. The process as claimed in claim 9 wherein said redox potential is between 150 mV and 500 mV.

19. The process as claimed in claim 11, wherein the ratio of the chemical oxygen demand (COD) of the lignocellulosic residues to the chemical oxygen demand (COD) of the starting aqueous mixture is between 5 and 20.

20. The process as claimed in claim 11, wherein the ratio of the chemical oxygen demand (COD) of the lignocellulosic residues to the chemical oxygen demand (COD) of the starting aqueous mixture is between 6 and 10.
